(19) United States
(12) Patent Application Publication
(10) Pub. No.: US 2024/0278037 A1
(43) Pub. Date: Aug. 22, 2024

(54) CATHETER APPARATUS FOR PHOTODYNAMIC THERAPY

(71) Applicants: THE ASAN FOUNDATION, Seoul (KR); UNIVERSITY OF ULSAN FOUNDATION FOR INDUSTRY COOPERATION, Ulsan (KR)

(72) Inventors: Do Hoon KIM, Seoul (KR); Jung-Hoon PARK, Hanam-si (KR); Daesung RYU, Seoul (KR)

(73) Assignees: THE ASAN FOUNDATION, Seoul (KR); UNIVERSITY OF ULSAN FOUNDATION FOR INDUSTRY COOPERATION, Ulsan (KR)

(21) Appl. No.: 18/564,790

(22) PCT Filed: May 16, 2022
(86) PCT No.: PCT/KR2022/006968

§ 371 Date: Nov. 28, 2023

(30) Foreign Application Priority Data
Jun. 3, 2021 (KR) 10-2021-0072383

(51) Int. Cl. A61N 5/06; A61F 2/90; A61F 2/95; A61N 5/067

(52) U.S. Cl. CPC A61N 5/062; A61F 2/90; A61F 2/95; A61N 5/067; A61F 2210/0076; A61F 2250/0067; A61N 2005/0643

(57) ABSTRACT

A catheter apparatus for photodynamic therapy includes: a catheter movable to a lesion site of tissues; a stent installed to be inserted into and retracted from an end of the catheter such that a length thereof is adjustable, and coated with a photosensitizer, and an optical fiber inserted into the catheter to be movable to be positioned at a central portion of the stent. The optical fiber irradiates the photosensitizer with a laser to activate the photosensitizer in a state where the stent is pulled out from the catheter and expanded.

### TECHNICAL FIELD

The present disclosure relates to a catheter apparatus for photodynamic therapy.

### BACKGROUND ART

In general, surgical procedures, stent placement, or drug-eluting stent procedures are performed to treat malignant strictures and tumors within the luminal organs.

However, in the case of traditional surgical procedures, a patient's recovery period is significantly longer, and there is a higher risk of complications, and on the other hand, stent placement and drug-eluting stent procedures offer the advantage of minimally invasive techniques resulting in a much shorter patient recovery period. Nonetheless, these procedures come with the challenge of potential restenosis and the risk of stent removal.

Meanwhile, research into photodynamic therapy for targeting cancer is currently underway, but it takes quite a long time to develop a photosensitizer that enables targeted therapy, and there are significant challenges in terms of both the development of the photosensitizer and regulatory approval.

### RELATED DOCUMENT

(Patent Document 1) Korean Patent No. 10-1650515 (Registered on Aug. 17, 2016)

### DETAILED DESCRIPTION OF THE DISCLOSURE

### Technical Problem

The embodiments of the present disclosure aim to provide a catheter apparatus for photodynamic therapy that can activate a photosensitizer coated on a stent to carry out photodynamic therapy for a lesion site.

### Technical Solution to the Problem

In accordance with an aspect of the present disclosure, there is provided a catheter apparatus for photodynamic therapy, the apparatus comprising: a catheter movable to a lesion site of tissues: a stent installed to be inserted into and retracted from an end of the catheter such that a length thereof is adjustable, and coated with a photosensitizer; and an optical fiber inserted into the catheter to be movable to be positioned at a central portion of the stent, wherein the optical fiber irradiates the photosensitizer with a laser to activate the photosensitizer in a state where the stent is pulled out from the catheter and expanded.

Further, the catheter apparatus may further include a guide wire, installed to be inserted into and retracted from the catheter, for guiding the catheter to the lesion site.

Further, the catheter may include: a support tube movable along the guide wire: an adjustment tube providing a tube passage through which the guide wire or the optical fiber moves, and supported on the support tube so that at least a portion of the stent is disposed outside the adjustment tube: a moving tube configured to accommodate at least a portion of the stent therein, and movable along the support tube to adjust an expansion length of the stent disposed outside the catheter; and a guide tip connected to an end of the adjustment tube, and communicating with the tube passage so that the guide wire or the optical fiber moves.

Further, the expansion length of the stent may be adjusted in proportion to a moving distance of the moving tube.

Further, when pressed by the moving tube, the stent may be positioned in a space between the adjusting tube and the moving tube to maintain a pressed state while enveloping the adjusting tube, and when pressure from the moving tube is released, the stent may expand and return to an original shape thereof.

Further, a rear end of the stent may be fixed to the catheter, and a front end of the stent may be fixed to the guide tip or provided in the form of a free end.

Further, the stent may be provided in the form of a cylindrical mesh that presses and envelops the adjusting tube under pressure from the moving tube in a space between the adjusting tube and the moving tube of the catheter.

Further, the catheter may further include a pusher connected to a rear end of the moving tube, and during a pull action of the pusher, the stent may expand from the adjusting tube of the catheter to come in close contact with the lesion site as pressure from the moving tube is released due to rearward movement of the moving tube, and during a push action of the pusher, the stent may be restored to the adjusting tube of the catheter due to pressure from the moving tube caused by forward movement of the moving tube.

Further, the photosensitizer may include one or more of Chlorin e6, Hematoporphyrin, Methylene blue, and Toluidine blue.

### Advantageous Effects of the Disclosure

In the embodiments of the present disclosure, a stent commonly used for non-vascular stenotic diseases is utilized in intraluminal expansion and coated with a photosensitizer to target a malignant stenotic disease within a luminal organ (such as the esophagus, duodenum, bile duct, pancreas, colon, small intestine, etc.) using a catheter apparatus, and this configuration of the stent allows eradication of cells, including cancer and stenotic diseases, through photodynamic therapy.

Furthermore, in the embodiments of the present disclosure, there is an advantage of contributing to treatment of malignant stenotic diseases by irradiating a photosensitizer coated on the catheter apparatus with a laser to activate the photosensitizer.

Furthermore, in the embodiments of the present disclosure, there is an advantage of being able to withdraw the catheter apparatus after photodynamic therapy, and adjusting a range of photodynamic therapy based on a length of the lesion.

### BRIEF DESCRIPTION OF THE DRAWING

FIG. 1 is a configuration view showing a catheter apparatus for photodynamic therapy according to an embodiment of the present disclosure.

FIG. 2 is an enlarged view showing part “A” of FIG. 1.

FIG. 3 is a configuration view showing a catheter apparatus for photodynamic therapy according to a modified example of the present disclosure.

FIG. 4 is a state view showing a state in which a guide wire is inserted into a lesion site in a catheter apparatus for photodynamic therapy according to an embodiment of the present disclosure.

FIG. 5 is a state view showing a state in which a catheter apparatus for photodynamic therapy according to an embodiment of the present disclosure is guided to a lesion site along a guide wire.

FIG. 6 is an enlarged view showing part “B” of FIG. 5.

FIG. 7 is a state view showing a state in which a stent of a catheter apparatus for photodynamic therapy according to an embodiment of the present disclosure is expanded at a lesion site.

FIG. 8 is a state view showing a state in which an optical fiber is moved inside a stent in a catheter apparatus for photodynamic therapy according to an embodiment of the present disclosure.

FIG. 9 is a state view showing a state in which an optical fiber irradiates a laser toward a photosensitizer of a stent to perform photodynamic therapy on a lesion site in the catheter apparatus for photodynamic therapy according to an embodiment of the present disclosure.

FIG. 10 is a state view showing a state in which a catheter apparatus for photodynamic therapy according to an embodiment of the present disclosure is withdrawn from the lesion site.

### BEST MODE FOR CARRYING OUT THE DISCLOSURE

Hereinafter, specific embodiments for implementing a spirit of the present disclosure will be described in detail with reference to the drawings.

In describing the present disclosure, detailed descriptions of known configurations or functions may be omitted to clarify the present disclosure.

When an element is referred to as being ‘connected’ to, ‘supported’ by, ‘accessed’ to, ‘supplied’ to, ‘transferred’ to, or ‘contacted’ with another element, it should be understood that the element may be directly connected to, supported by, accessed to, supplied to, transferred to, or contacted with another element, but that other elements may exist in the middle.

The terms used in the present disclosure are only used for describing specific embodiments, and are not intended to limit the present disclosure. Singular expressions include plural expressions unless the context clearly indicates otherwise.

Further, in the present disclosure, it is to be noted that expressions, such as the upper side and the lower side, are described based on the illustration of drawings, but may be modified if directions of corresponding objects are changed. For the same reasons, some components are exaggerated, omitted, or schematically illustrated in the accompanying drawings, and the size of each component does not fully reflect the actual size.

Terms including ordinal numbers, such as first and second, may be used for describing various elements, but the corresponding elements are not limited by these terms. These terms are only used for the purpose of distinguishing one element from another element.

In the present specification, it is to be understood that the terms such as “including” are intended to indicate the existence of the certain features, areas, integers, steps, actions, elements, combinations, and/or groups thereof disclosed in the specification, and are not intended to preclude the possibility that one or more other certain features, areas, integers, steps, actions, elements, combinations, and/or groups thereof may exist or may be added.

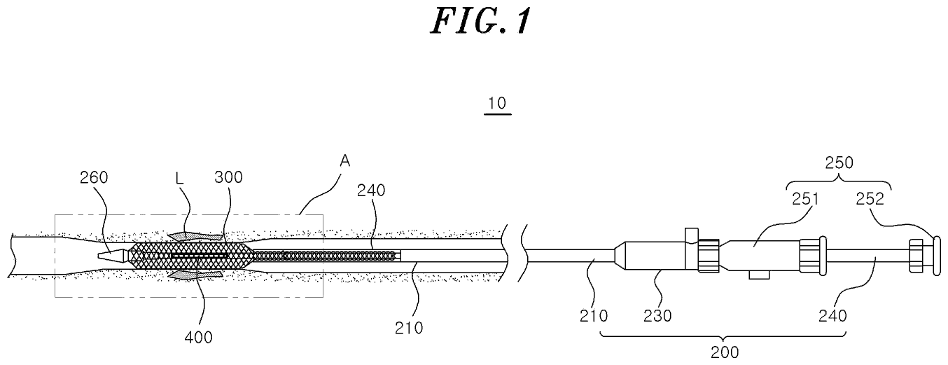

Hereinafter, the specific configuration of a catheter apparatus for photodynamic therapy according to an embodiment of the present disclosure will be described with reference to FIGS. 1 to 10.

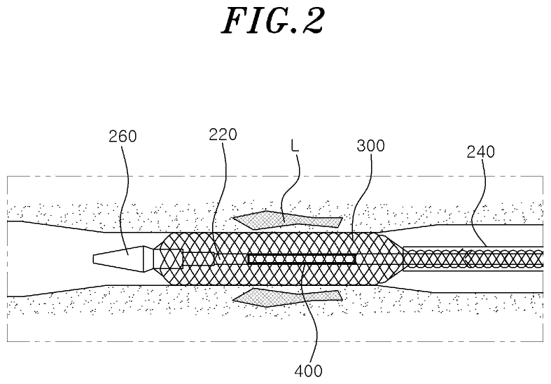

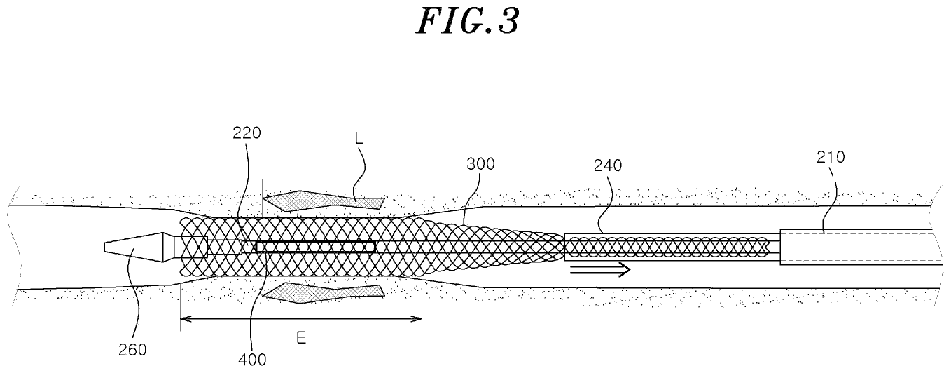

FIG. 1 is a configuration view showing a catheter apparatus for photodynamic therapy according to an embodiment of the present disclosure, FIG. 2 is an enlarged view showing part “A” of FIG. 1, and FIG. 3 is a configuration view showing a catheter apparatus for photodynamic therapy according to a modified example of the present disclosure.

As shown in FIGS. 1 and 2, a catheter apparatus 10 for photodynamic therapy according to an embodiment of the present disclosure may include a guide wire 100, a catheter 200, and a stent 300.

Specifically, the guide wire 100 may be provided in the form of a wire to guide the catheter 200 to a lesion site L occurring in a luminal organ (such as the esophagus, duodenum, bile duct, pancreas, colon, small intestine, etc.) with a malignant stenotic disease. The guide wire 100 may be inserted into and mounted on the catheter 200 so as to move through the catheter 200. For example, the guide wire 100 may be a guide wire that is movable to the lesion site L.

The catheter 200 may be guided by the guide wire 100 to move to the lesion site L of tissues. With the catheter 200 positioned at the lesion site L of tissues, the guide wire 100 may be separated from the catheter 200, the stent 300 may expand toward the lesion site L of tissues, and an optical fiber 400 may be inserted into the catheter 200 and positioned close to the lesion site L of tissues.

The catheter 200 may include a support tube 210, an adjustment tube 220, a sheath hub 230, a moving tube 240, and a pusher 250. The support tube 210 may be provided in the form of a hollow tube. The adjustment tube 220 may be provided inside the support tube 210. The sheath hub 230 may be coupled to a rear end of the support tube 210.

The adjustment tube 220 may be provided in the form of a hollow tube. The adjustment tube 220 may be installed through the support tube 210. In the adjustment tube 220, a tube passage may be provided through which the guide wire 100 or the optical fiber 400 can be inserted and moved. The stent 300 may be mounted at a circumferential portion of a front end side of the adjustment tube 220. In addition, a conical guide tip 260 may be coupled to a front end of the adjustment tube 220. The sheath hub 230 may be coupled to a rear end of the adjustment tube 220.

The sheath hub 230 may be coupled to a rear end of the support tube 210. The sheath hub 230 may be provided in the form of a hollow tube through which the moving tube 240 passes.

The moving tube 240 may be positioned between the support tube 210 and the adjusting tube 220 to be movable in a longitudinal direction. For example, an inner diameter of the moving tube 240 may be larger than an outer diameter of the adjustment tube 220, and an outer diameter of the moving tube 240 may be smaller than an inner diameter of the support tube 210. A front end portion of the moving tube 240 may be provided in the form of a tube that selectively envelops the stent 300.

A rear end portion of the moving tube 240 may be connected to the pusher 250. During a pull or push action of the pusher 250, the moving tube 240 may be moved backward or forward along the support tube 210. The moving tube 240 may be made of a transparent or translucent material. When the moving tube 240 is moved backwards (in a direction in which a pusher pulls) along the support tube 210, the stent 300 may expand while moving away from the moving tube 240.

The pusher 250 may include a pusher cap 251 coupled to a rear end of the sheath hub 230, and an adjustment cap 252 connected to a rear end of the moving tube 240 to move the moving tube 240. When the adjustment cap 252 is moved in a direction away from the pusher cap 251 during a pull action of the pusher 250, the moving tube 240 may be moved backward along the support tube 210. When the pusher 250 is pushed and the adjustment cap 252 is moved in a direction closer to the pusher cap 251, the moving tube 240 may be moved forward along the support tube 210.

The stent 300 may be provided in the form of a cylindrical or pouch-shaped mesh. The stent 300 may be expanded from an end portion of the catheter 200 such that a length thereof is adjustable, or may be restored in the end portion of the catheter 200.

For example, when the stent 300 is pressed by the moving tube 240 due to forward movement of the moving tube 240, the stent 300 is positioned in a space between the adjustment tube 220 and the moving tube 240, and this allows the stent 300 to be enveloped by the adjustment tube 220 and maintained in a pressed state. Also, when the pressure from the moving tube 240 on the stent 300 is released due to backward movement of the moving tube 240, the stent 300 may expand and return to its original mesh shape.

At this point, a rear end portion (distal part) of the stent 300 may be fixed to the adjustment tube 220 of the catheter 200, and the front end portion (proximal part) of the stent 300 may be fixed to the guide tip 260 of the catheter 200. As such, when the rear end portion and the front end portion of the stent 300 are fixed to the catheter 200, the stent 300 is placed in the center region of tissues, making focused photodynamic therapy possible.

Referring to FIG. 3, in the stent 300 according to a modified example of the present disclosure, the rear end portion (distal part) of the stent 300 may be fixed to the adjustment tube 220 of the catheter 200, and the front end portion (proximal part) of the stent 300 may be provided in the form of a free end that is not fixed to the guide tip 260 of the catheter 200. In this way, if the front end portion of the stent 300 is provided in the form of a free end to the catheter 200, the stent 300 may be placed over a wider range, making extensive photodynamic therapy possible.

An expansion length E of the expanded stent 300 may be adjusted depending on a moving distance by which the moving tube 240 moves. For example, when the moving tube 240 is moved backward (in a direction to be pulled by the pusher), the expansion length E of the expanded stent 300 may increase in proportion with a moving distance of the moving tube 240. Also, when the moving tube 240 is moved forward (in a direction to be pushed by the pusher), the stent 300 may be restored by the moving tube 240 and the expansion length E of the stent 300 may be reduced.

The stent 300 may be coated with a photosensitizer. The photosensitizer may include a material capable of photodynamic therapy for a lesion site of tissues when irradiated with a laser. For example, the photosensitizer may include at least one of Chlorin e6, Hematoporphyrin, Methylene blue, or Toluidine blue.

The coating of the photosensitizer may be applied as a single layer on the stent 300. A thickness of coating of the photosensitizer may be thinner than a wire diameter of the stent 300. The photosensitizer may be evenly coated on a surface of the stent 300. However, when the stent 300 is installed at a coating jig required for coating, the coating is applied to the stent 300 in proximity to the coating jig, and as a result, the photosensitizer may be coated more on the inner side than the outer side of the stent 300. However, due to the nature of the stent 300, when the stent 300 is positioned at a lesion site of tissues, the stent 300 tends to deform to match a shape of the tissues, and due to the nature of the lesion tissues, the lesion tissues tend to move from the outer side to the inner side of the stent 300, and thus, even if the photosensitizer is coated more on the inner side than on the outer side of the stent 300, the concerns regarding photodynamic therapy effectiveness are minimal.

The optical fiber 400 may be inserted into and retracted from the catheter 200 so as to be positioned at a center portion of the stent 300. For example, the optical fiber 400 may be a cylindrical diffuser. The optical fiber 400 may be electrically connected to a laser supply device (not shown) to irradiate the stent 300 with a laser. The laser supply device may adjust the wavelength, irradiation intensity, and irradiation interval of the laser according to the characteristics of the lesion site of tissues.

The optical fiber 400 may be moved in the adjustment tube 220. To this end, an outer diameter of the optical fiber 400 may be smaller than an inner diameter of the adjustment tube 220. Of course, the outer diameter of the optical fiber 400 may be smaller than the inner diameter of the moving tube 240.

Consequently, in a state where the stent 300 is pulled out from the catheter 200 and expanded, the optical fiber 400 may irradiate the photosensitizer coated on the stent 300 with a laser received from the laser supply device, thereby activating the photosensitizer.

Hereinafter, the operation and effects of the catheter apparatus for photodynamic therapy with the above-described configuration will be described.

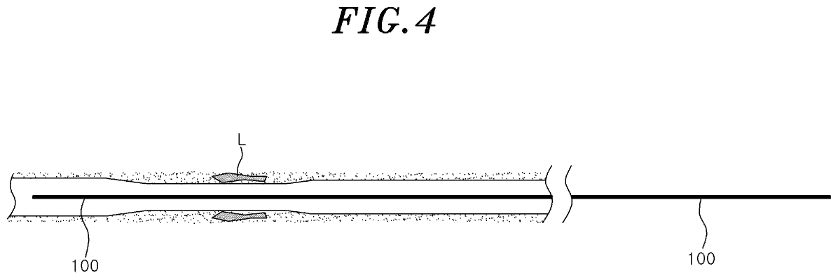

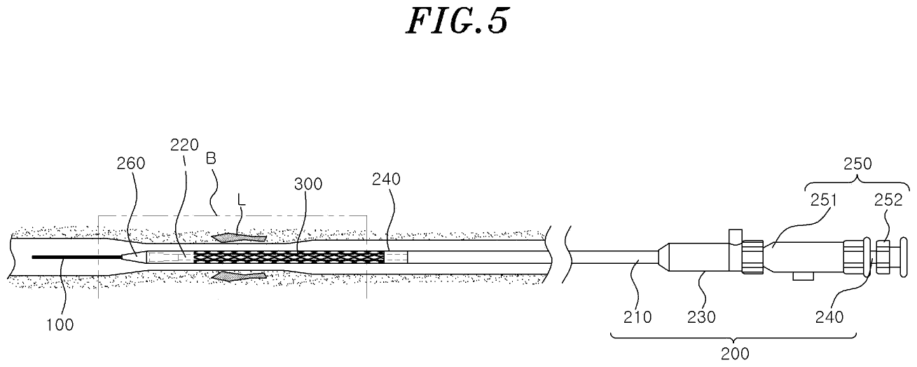

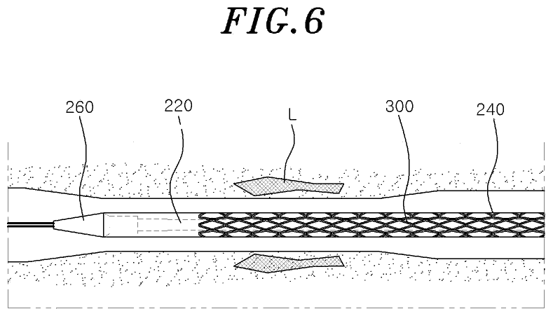

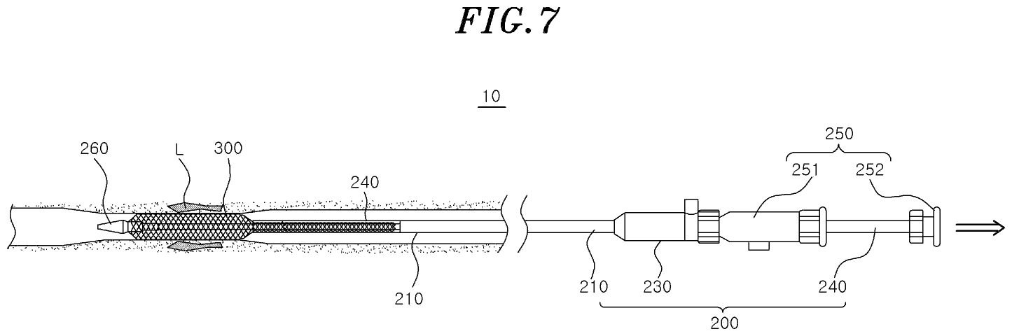

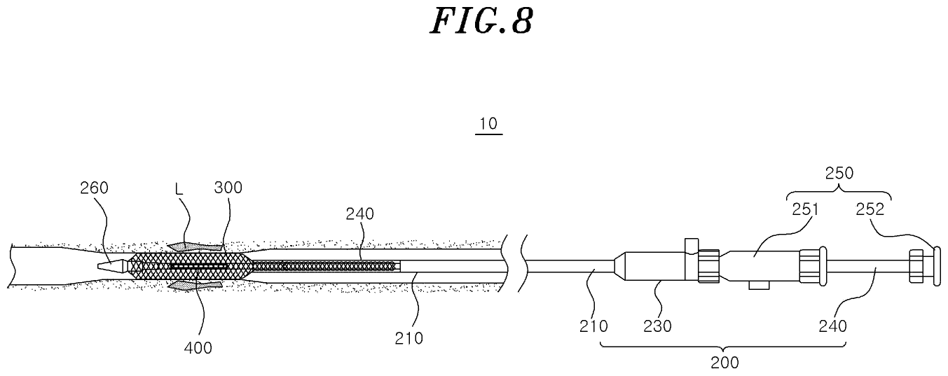

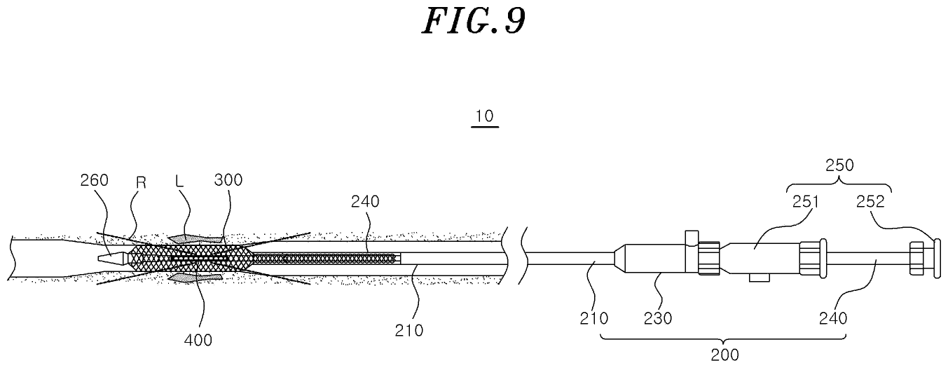

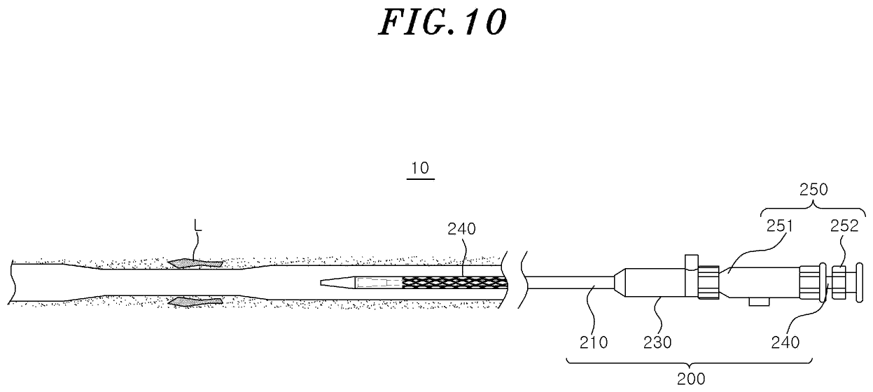

FIG. 4 is a state view showing a state in which a guide wire is inserted into a lesion site in a catheter apparatus for photodynamic therapy according to the embodiment of the present disclosure: FIG. 5 is a state view showing a state in which the catheter apparatus for photodynamic therapy according to the embodiment of the present disclosure is guided to a lesion site along a guide wire: FIG. 6 is an enlarged view showing part “B” of FIG. 5; FIG. 7 is a state view showing a state in which a stent of the catheter apparatus for photodynamic therapy according to the embodiment of the present disclosure is expanded at the lesion site; FIG. 8 is a state view showing a state in which an optical fiber is moved inside the stent in the catheter apparatus for photodynamic therapy according to the embodiment of the present disclosure: FIG. 9 is a state view showing a state in which the optical fiber irradiates a laser toward a photosensitizer of the stent to perform photodynamic therapy on the lesion site in the catheter apparatus for photodynamic therapy according to the embodiment of the present disclosure; and FIG. 10 is a state view showing a state in which the catheter apparatus for photodynamic therapy according to the embodiment of the present disclosure is withdrawn from the lesion site.

As shown in FIG. 4, first, the guide wire 100 is moved to a lesion site L occurring in tissues of a luminal organ with a malignant stenotic disease.

As shown in FIGS. 5 and 6, when the guide wire 100 is positioned at the lesion site L of the tissues, the catheter 200 is moved to the lesion site L of the tissues through the guide wire 100 to position the stent 300 to correspond to the lesion site L of the tissues.

As shown in FIG. 7, when the stent 300 is positioned to correspond to the lesion site L of the tissues, the stent 300 expands to adhere to the lesion site L of the tissues and the guide wire 100 is separated from the catheter 200. For example, in a state where the stent 300 is positioned to correspond to the lesion site L of the tissues, if the pressure from the moving tube 240 on the stent 300 is released by a pull action of the pusher 250, the stent 300 expands to adhere to the lesion site L of the tissues.

As shown in FIG. 8, if the stent 300 adheres to the lesion site L of the tissues due to expansion thereof, the optical fiber 400 is moved to the lesion site L of the tissues through the catheter 200. At this point, the optical fiber 400 may be positioned at a center portion of the unfolded stent 300.

As shown in FIG. 9, when the optical fiber 400 is positioned at the lesion site L of the tissues through the catheter 200, a laser R is directed onto the lesion site L of the tissues via the optical fiber 400, activating the photosensitizer coated on the stent 300 to perform photodynamic therapy.

As shown in FIG. 10, when irradiation of the laser toward the lesion site L is completed, the stent 300 is restored and separated. At this point, as the stent 300 is pressed by the moving tube 240 due to a push action of the pusher 250, the stent 300 may be restored in a space between the moving tube 240 and the support tube 210 of the catheter 200.

As described above, in the present disclosure, it is possible to eradicate cells in conditions like cancer and stenotic diseases through photodynamic therapy, to contribute to the treatment of malignant stenotic diseases through the photodynamic therapy by irradiating a laser onto a photosensitizer coated on a catheter apparatus to activate the photosensitizer, to withdraw the catheter apparatus after photodynamic therapy, and to allow adjustment of a photodynamic therapy range according to a length of the lesion.

The embodiments described above merely illustrate some examples of the present technical idea, the scope of the present technical idea is not limited to the described embodiments, those skilled in the art can variously modify, change, or substitute within the scope of the present technical idea, which should be construed as belonging to the scope of the present technical idea

1. A catheter apparatus for photodynamic therapy, the apparatus comprising:
a catheter movable to a lesion site of tissues;
a stent installed to be inserted into and retracted from an end of the catheter such that a length thereof is adjustable, and coated with a photosensitizer; and
an optical fiber inserted into the catheter to be movable to be positioned at a central portion of the stent,
wherein the optical fiber irradiates the photosensitizer with a laser to activate the photosensitizer in a state where the stent is pulled out from the catheter and expanded.

2. The catheter apparatus of claim 1, further comprising:
a guide wire, installed to be inserted into and retracted from the catheter, for guiding the catheter to the lesion site.

3. The catheter apparatus of claim 2, wherein the catheter comprises:
a support tube movable along the guide wire;
an adjustment tube providing a tube passage through which the guide wire or the optical fiber moves, and supported on the support tube so that at least a portion of the stent is disposed outside the adjustment tube;
a moving tube configured to accommodate at least a portion of the stent therein, and movable along the support tube to adjust an expansion length of the stent disposed outside the catheter; and
a guide tip connected to an end of the adjustment tube, and communicating with the tube passage so that the guide wire or the optical fiber moves.

4. The catheter apparatus of claim 3, wherein the expansion length of the stent is adjusted in proportion to a moving distance of the moving tube.

5. The catheter apparatus of claim 3, wherein:
when pressed by the moving tube, the stent is positioned in a space between the adjustment tube and the moving tube to maintain a pressed state while enveloping the adjustment tube, and
when pressure from the moving tube is released, the stent expands and returns to an original shape thereof.

6. The catheter apparatus of claim 3, wherein:
a rear end of the stent is fixed to the catheter, and
a front end of the stent is fixed to the guide tip or provided in the form of a free end.

7. The catheter apparatus of claim 3, wherein:
the stent is provided in the form of a cylindrical mesh that presses and envelops the adjustment tube under pressure from the moving tube in a space between the adjustment tube and the moving tube of the catheter.

8. The catheter apparatus of claim 7, wherein:
the catheter further comprises a pusher connected to a rear end of the moving tube, and
during a pull action of the pusher, the stent expands from the adjustment tube of the catheter to come in close contact with the lesion site as pressure from the moving tube is released due to rearward movement of the moving tube, and during a push action of the pusher, the stent is restored to the adjustment tube of the catheter due to pressure from the moving tube caused by forward movement of the moving tube.

9. The catheter apparatus of claim 1, wherein the photosensitizer comprises one or more of Chlorin e6, Hematoporphyrin, Methylene blue, and Toluidine blue.
